(19) United States
(12) Patent Application Publication
(10) Pub. No.: US 2025/0345508 A1
(43) Pub. Date: Nov. 13, 2025

(54) ASPIRATION FLOW CONTROL VALVE

(71) Applicant: KONINKLIJKE PHILIPS N.V., EINDHOVEN (NL)

(72) Inventor: Scott Robert WILSON, MAPLE GROVE, MN (US)

(21) Appl. No.: 18/871,189

(22) PCT Filed: Jun. 1, 2023
(86) PCT No.: PCT/EP2023/064683

§ 371 Date: Dec. 3, 2024

Related U.S. Application Data
(60) Provisional application No. 63/350,446, filed on Jun. 9, 2022.

(51) Int. Cl. A61M 1/00; A61M 39/22; F16K 31/122

(52) U.S. Cl. CPC A61M 1/743; A61M 1/60; A61M 39/227; F16K 31/1221; A61M 2039/226; A61M 2210/12

(57) ABSTRACT

An aspiration system (1) for an intravascular device includes an aspiration catheter (2) configured for insertion into an associated blood vessel: and a normally closed flow control valve (10) connected to control the aspiration of material from the associated blood vessel via the aspiration catheter. The normally closed flow control valve includes a flow inlet (14) operatively connected with the aspiration catheter, a flow outlet (16) operatively connectable with an associated vacuum pump, and a vacuum pull actuator (18) operatively connected to the flow outlet whereby vacuum at the flow outlet operates the vacuum pull actuator to open the normally closed flow control valve.

### FIELD

The following relates generally to the catheter arts, intravascular therapy arts, aspiration catheter arts, lesion treatment arts, and related arts.

### BACKGROUND

Catheter based aspiration devices are used to remove blood clots and/or thrombus from arterial and venous vasculature. In the venous system, aspiration catheters are used to treat deep vein thrombosis (DVT) and/or pulmonary embolism, and are typically larger in diameter, as the vessel size is larger, and the clot/thrombus is larger.

The use of larger bore (diameter) catheters increases the risk for blood loss. The large diameter catheter combined with high vacuum can remove a large volume of blood in a short timeframe if not controlled. Some devices incorporate a manual on/off valve, which requires the user to sense when blood is being aspirated, and then manually shut the valve. Other systems, such as those made by Penumbra Lightning Inc. (Alameda, CA. USA), incorporate a computer controlled system to monitor pressures, and shut the valve when vacuum level drops below a pre-determined threshold. However, this system requires capital equipment, and many clinics cannot afford the cost of the system.

When aspirating clot or thrombus from blood vessels, typically large bore catheters are used with high level of suction. If the distal end of the catheter becomes dislodged from the clot, a large volume of blood can be aspirated from the patient. Aspiration catheters typically have a manual shut-off valve that requires the user to shut the valve if they notice blood being aspirated. However, if uncontrolled, flow rate can be 300-1000 milliliters (mL) per minute.

The following discloses certain improvements to overcome these problems and others.

### SUMMARY

In some embodiments disclosed herein, an aspiration system for an intravascular device includes an aspiration catheter configured for insertion into an associated blood vessel; and a normally closed flow control valve connected to control the aspiration of material from the associated blood vessel via the aspiration catheter. The normally closed flow control valve includes a flow inlet operatively connected with the aspiration catheter, a flow outlet operatively connectable with an associated vacuum pump, and a vacuum pull actuator operatively connected to the flow outlet whereby vacuum at the flow outlet operates the vacuum pull actuator to open the normally closed flow control valve.

In some embodiments disclosed herein, an aspiration system includes an aspiration catheter configured for insertion into an associated blood vessel; and a normally closed flow control valve having an inlet operatively connected with the aspiration catheter and an outlet operatively connectable with an associated vacuum pump. The control valve includes a main valve body; and a biasing member disposed within an interior of the main valve body. When a vacuum level at the outlet of the control valve is equal to or exceeds a predetermined vacuum threshold, biasing provided by the biasing member is overcome to initiate opening of the control valve.

In some embodiments disclosed herein, an intravascular treatment aspiration method includes inserting an aspiration catheter into a blood vessel; applying a vacuum to the aspiration catheter using a vacuum pump and with a vacuum-actuated valve in-line between the aspiration catheter and the vacuum pump; opening the vacuum-actuated valve in response to the applied vacuum generating a vacuum level at a vacuum-actuated valve that is equal to or exceeds a predetermined vacuum threshold to initiate aspiration of material in the blood vessel via the aspiration catheter; and during the aspiration, closing the vacuum-actuated valve in response to the vacuum level at the vacuum-actuated valve decreasing below the predetermined vacuum threshold to stop the aspiration of material in the blood vessel via the aspiration catheter.

One advantage resides in providing a controllable valve for automatically reducing or shutting off suction during an intravascular therapy procedure in response to a loss of contact with the clot or other material being aspirated.

Another advantage resides in providing an inexpensive automatic valve for reducing or shutting off suction during an intravascular therapy procedure in response to a loss of contact with the clot or other material being aspirated.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIGS. 1-3 diagrammatically illustrate an aspiration system in accordance with the present disclosure.

FIGS. 4A-4C diagrammatically illustrate embodiments of configurations of a valve of the system of FIG. 1.

FIG. 5 diagrammatically illustrates a method of performing an aspiration method using the device of FIG. 1.

### DETAILED DESCRIPTION

In endovascular procedures that employ aspiration, the goal is to use the aspiration to draw clot material out of the target area after it has been debulked by laser ablation, mechanical cutting, ultrasonic disruption, or another technique. However, if the aspiration orifice at the distal end of the instrument becomes disengaged with clot material, then it will begin aspirating blood, which can quickly produce undesirable blood loss from the patient.

To address this problem, existing instruments include a manual shutoff valve. However, the physician may fail to react in a timely manner, thus still resulting in undesirable blood loss.

The following discloses a passive vacuum controlled valve for protecting against excessive blood loss. The valve is normally closed (for example, by a biasing spring). During aspiration against clot material, the vacuum pump draws a strong vacuum, and this is tapped to evacuate a plenum (e.g., a piston cylinder) containing a diaphragm (e.g., a piston), thus pulling the diaphragm to open the valve. Advantageously, this is a purely passive operation driven by the vacuum.

In a variant embodiment, the valve comprises a shaft with a teardrop-shaped opening that aligns with the main fluid passage of the valve when in the open state. In the off state, the tip of the teardrop-shaped opening still overlaps the main fluid passage so as to provide a trickling blood flow, which can be beneficial as the surgeon may look for some flow to verify proper operation of the aspiration system.

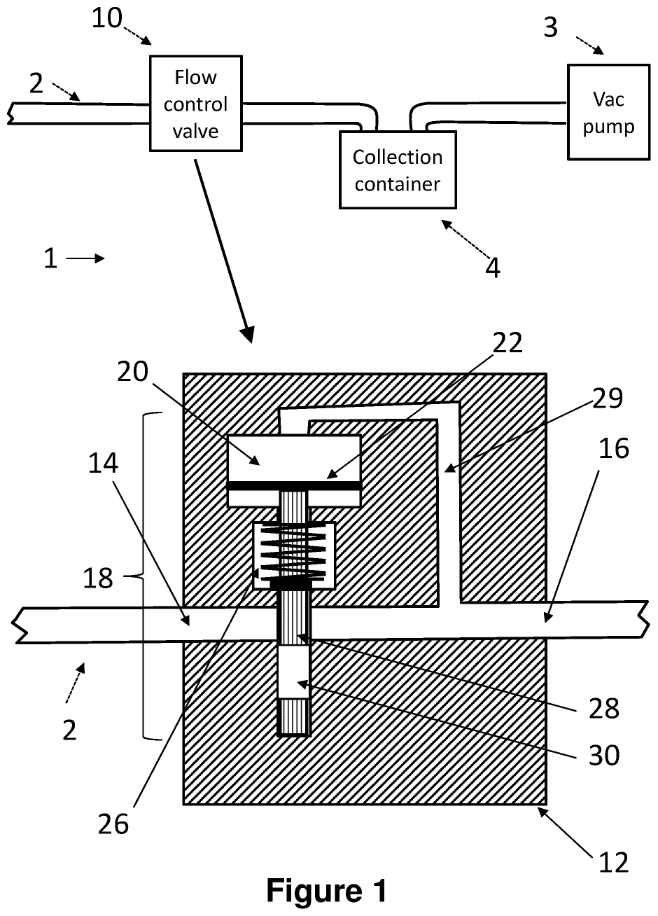

With reference to FIG. 1, an illustrative catheter-based aspiration system or apparatus 1 is diagrammatically shown. As shown in FIG. 1, the apparatus 1 includes an aspiration catheter 2 configured for insertion into an associated blood vessel. A vacuum source or pump 3 is operatively connected with the aspiration catheter 2 to aspirate material from the blood vessel via the aspiration catheter 2 by supplying aspiration in the blood vessel. In the illustrative example, this operative connection is by way of an intervening collection container 4. The device may further include a therapy device (not shown) for debulking a clot or other vascular buildup. The therapy device may, for example, comprise an ablation laser, mechanical cutter, ultrasonic transducer, or so forth. In a typical use case, the therapy device debulks the clot and the aspiration removes the debulked material.

A normally closed flow control (or vacuum-actuated) valve 10 is connected to control the aspiration of material from the blood vessel via the aspiration catheter 2. The lower drawing of FIG. 1 shows the control valve 10 in the normally closed position by way of a side sectional view. The control valve 10 includes a main valve body 12 defining a flow inlet 14 operatively connected with the aspiration catheter 2, and a flow outlet 16 operatively connected with the vacuum pump 3. The collection container 4 is interposed in a flow path from the outlet 16 of the control valve 10 to the vacuum pump 3, whereby material aspirated from the blood vessel via the aspiration catheter 2 collects in the collection container 4. In one example, the collection container 4 is disposed in-line between the vacuum-actuated valve 10 and the vacuum pump 3 (as shown in FIG. 1). In another example, the collection container 4 is disposed in-line between the vacuum-actuated valve 10 and the aspiration catheter 2. In yet another example, the vacuum-actuated valve 10 could be integrated into the catheter 2.

A vacuum pull actuator 18 of the control valve 10 is operatively connected to the flow outlet 16, whereby vacuum at the flow outlet 16 operates the vacuum pull actuator 18 to open the normally closed flow control valve 10. As shown in FIG. 1, the vacuum pull actuator 18 includes a piston assembly 20, 22, 28 including a piston cylinder 20 disposed in the main valve body 12, piston or diaphragm 22, and a piston rod or pin 28. A biasing member 26, such as an illustrative spring 26, biases the piston assembly 20, 22, 28 into a closed position. A vacuum applied to the piston cylinder 20 operates to open the valve. By way of a fluid path 29 connecting the piston cylinder 20 to the outlet 16, presence of a vacuum at the outlet 16 overcomes the spring force applied by the spring 26 to lift the piston 22 and open the valve.

The spring 26 has a spring constant that is stiff enough to keep the valve normally closed, but flexible enough to allow vacuum in the vacuum cylinder 20 to overcome the force applied by the spring 26 to thereby lift the piston 22. The piston rod 28 is connected with the piston 22, and includes a through-hole or opening 30.

The spring 26 is thus arranged to bias the piston assembly 20, 22, 28 to a closed position (shown in FIG. 1) in which the through-hole 30 of the piston rod 28 is misaligned with a fluid flow path from the flow inlet 14 to the flow outlet 16. The opening 30 of the piston rod 28 is configured to not align with the diameter of the interior of the aspiration catheter 2. That is, a portion of the flow path through the aspiration catheter 2 is blocked by the piston rod 28.

In some examples, the through-hole 30 of the piston rod 28 has a teardrop shape configured to at least partially align with the diameter of the interior of the aspiration catheter 2. A tip of the teardrop is aligned with the fluid flow path when the piston assembly is in the closed position. A nonzero trickle flow path from the flow inlet 14 to the flow outlet 16 is provided in the closed position of the normally closed flow control valve 10.

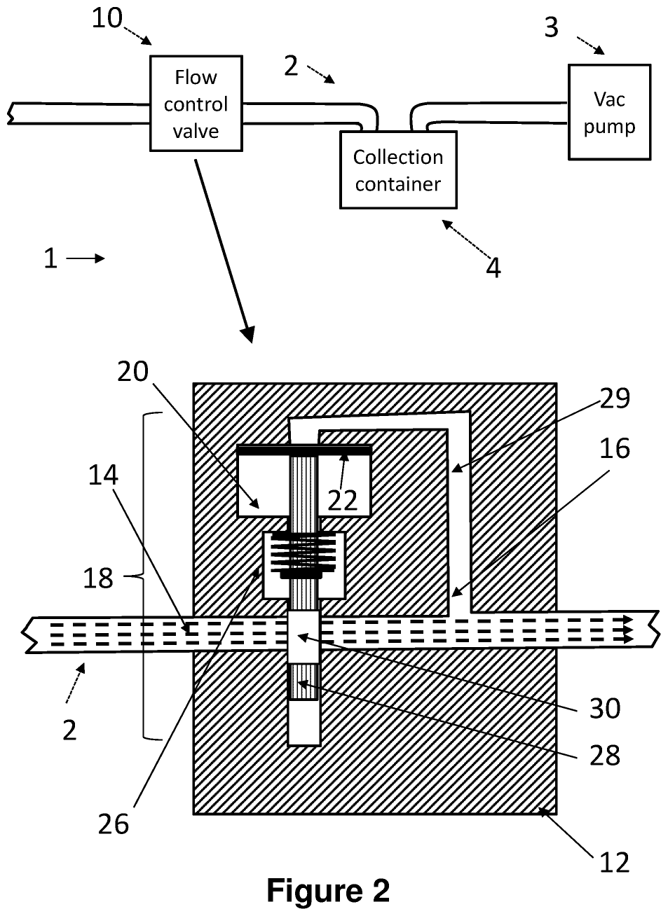

On the other hand, in an open position, when the vacuum level at the outlet 16 of the valve 10 is equal to or exceed the predetermined vacuum threshold, this vacuum is transferred to the piston cylinder 20 by way of the fluid path 29 connecting the piston cylinder 20 to the outlet 16, and the vacuum in the piston cylinder 20 lifts the piston or diaphragm 22 against the force applied by the spring 26 to initiate opening of the control valve 10. The open configuration of the control valve 10 is shown in FIG. 2. The piston cylinder 20 is operatively connected to the flow outlet 16 by the fluid path 29. Vacuum at the flow outlet 16 thus moves the piston assembly to an open position in which the through-hole 30 of the piston rod 28 is aligned with the fluid flow path. As shown in FIG. 2, the opening 30 is configured to align with an interior of the aspiration catheter 2 when the control valve 10 is in a fully open configuration.

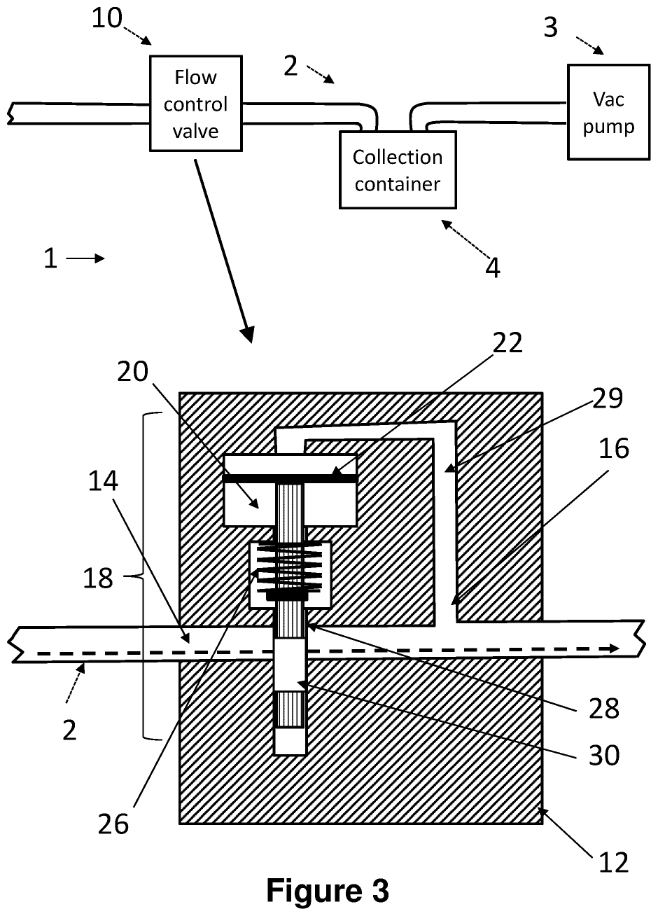

FIG. 3 shows a partially-open configuration of the control valve 10, in which a portion of the through-hole 30 of the piston rod 28 is partially aligned with the diameter of the aspiration catheter 2. In this manner, the control valve 10 can control the flow rate of the aspiration through the aspiration catheter 2. The vacuum pump 2 is on, but is aspirating blood with a low viscosity, so the supplied vacuum is weak, and blood may be beginning to flow through the catheter 2. The weakened vacuum is transferred to the piston cylinder 20 thus reducing the force countering the spring force provided by the biasing spring 26, causing the spring to lower the piston 22 and hence lower the through-hole 30.

In summary, the vacuum from the vacuum pump 3 operates to open the vacuum-actuated control valve 10. So long as the clot being aspirated presents sufficient resistance to enable the vacuum pump to maintain vacuum, the valve 10 will be open to provide aspiration of the clot. If blood (or lower viscosity fluid) is aspirated, this reduces the vacuum and the valve 10 will shut to a small lumen size (or completely close, depending on the configuration of the through-hole 30), such that only a small volume of blood (or no blood) will be aspirated until the catheter 2 is again positioned against clot material, at which time the vacuum level will increase and the vacuum-actuated control valve 10 re-opens.

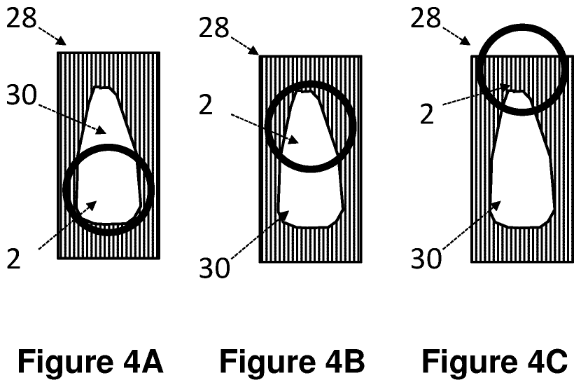

FIGS. 4A-C show the position of the through-hole 30 respective to the fluid flow path of the catheter 2 for different examples of the configurations of the control valve 10. FIG. 4A shows the open configuration, in which the widest portion of the teardrop shaped through-hole 30 is aligned with the diameter of the aspiration catheter 2 (diagrammatically shown as a circle). FIG. 4B shows the partially-open configuration, in which the teardrop shape of the through-hole 30 is partially aligned with the diameter of the aspiration catheter 2. FIG. 4C shows the closed configuration, in which the teardrop shape of the through-hole 30 is not aligned with the diameter of the aspiration catheter 2. However, in the closed configuration, the illustrative tear-shaped through-hole 30 still allows some leakage flow, as shown in the portion of the through-hole 30 overlapping the aspiration catheter 2. When the catheter 2 is against a viscous substance, such as a clot, a high vacuum level will be maintained (i.e., 20-25 inHg), and the valve 10 will remain in the open position. If a lower viscosity fluid, such as blood, is being aspirated, the vacuum level will drop, and the piston rod 28 slide to a partially closed position. The blood flow will still keep the vacuum level partially elevated (i.e., 3-15 inHg).

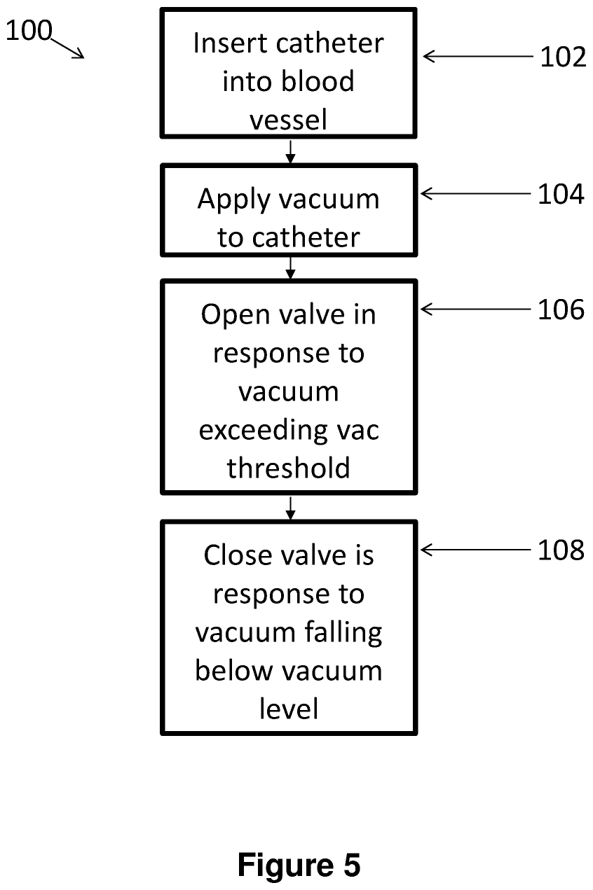

Referring to FIG. 5, an illustrative embodiment of an endovascular therapy method 100 using the aspiration apparatus 1 is diagrammatically shown as a flowchart. At an operation 102, the aspiration catheter 2 is inserted into the blood vessel. At an operation 104, a vacuum is applied to the aspiration catheter using the vacuum pump 3. At an operation 106, the valve 10 is opened in response to the applied vacuum generating a vacuum level at the valve 10 that is equal to or exceeds a predetermined vacuum threshold to initiate aspiration of material in the blood vessel via the aspiration catheter 2. With the valve open, material is aspirated. The aspirated material can be collected in the collection container 4. At an operation 108, during the aspiration, the vacuum-actuated valve 10 can automatically close in response to the vacuum level at the vacuum-actuated valve 10 decreasing below the predetermined vacuum threshold to stop the aspiration of material in the blood vessel via the aspiration catheter 2. For example, operation 108 will automatically occur if the tip of the catheter 2 inadvertently disengages from the clot so that blood begins to be aspirated.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An aspiration system for an intravascular device, the aspiration system comprising:
an aspiration catheter configured for insertion into an associated blood vessel; and
a normally closed flow control valve connected to control the aspiration of material from the associated blood vessel via the aspiration catheter, the normally closed flow control valve including:
a flow inlet operatively connected with the aspiration catheter,
a flow outlet operatively connectable with an associated vacuum pump, and
a vacuum pull actuator operatively connected to the flow outlet whereby vacuum at the flow outlet operates the vacuum pull actuator to open the normally closed flow control valve.

2. The aspiration system of claim 1, wherein the vacuum pull actuator includes:
a piston cylinder;
a piston assembly including a piston disposed in the piston cylinder and a piston rod connected with the piston, the piston rod having a through-hole; and
a spring arranged to bias the piston assembly to a closed position in which the through-hole of the piston rod is misaligned with a fluid flow path from the flow inlet to the flow outlet;
wherein the piston cylinder is operatively connected to the flow outlet, whereby vacuum at the flow outlet moves the piston assembly to an open position in which the through-hole of the piston rod is aligned with the fluid flow path.

3. The aspiration system of claim 2, wherein the through-hole of the piston rod-has a teardrop shape and a tip of the teardrop is aligned with the fluid flow path when the piston assembly is in the closed position whereby a nonzero trickle flow path from the flow inlet-to the flow outlet-is provided in the closed position of the normally closed flow control valve.

4. The aspiration system of claim 1, further comprising:
a collection container interposed in a flow path from the outlet of the normally closed flow control valve to the associated vacuum pump, whereby material aspirated from the associated blood vessel via the aspiration catheter collects in the collection container.

5. An aspiration system, comprising:
an aspiration catheter configured for insertion into an associated blood vessel; and
a normally closed flow control valve-having an inlet operatively connected with the aspiration catheter and an outlet operatively connectable with an associated vacuum pump, the control valve comprising:
a main valve body; and
a biasing member disposed within an interior of the main valve body;
wherein when a vacuum level at the outlet of the control valve is equal to or exceeds a predetermined vacuum threshold, biasing provided by the biasing member is overcome to initiate opening of the control valve.

6. The system of claim 5, wherein the control valve further comprises:
a vacuum-actuated piston assembly that is normally closed by force applied by the biasing member,
wherein when the vacuum level at the outlet of the control valve is equal to or exceeds the predetermined vacuum threshold the force applied by the biasing member is overcome by the vacuum-actuated piston assembly to initiate opening of the control valve.

7. The system of claim 6, wherein a piston cylinder of the vacuum-actuated piston assembly is connected with the outlet of the control valve.

8. The system of claim 5, wherein:
a piston rod of the vacuum-actuated piston assembly includes an opening configured to align with an interior of the aspiration catheter when the control valve-is in a fully open configuration.

9. The system of claim 8, wherein:
when the control valve-is in the normally closed configuration, the opening of the piston rod is configured to not align with the diameter of the interior of the aspiration catheter.

10. The system of claim 8, wherein the opening comprises a tear-drop shape configured to at least partially align with the diameter of the interior of the aspiration catheter.

11. The system of claim 5, wherein the biasing member comprises a spring.

12. The system of claim 5, further comprising:
a vacuum source configured to supply aspiration in the blood vessel.

13. An intravascular treatment aspiration method, comprising:
inserting an aspiration catheter-into a blood vessel;
applying a vacuum to the aspiration catheter using a vacuum pumpand with a vacuum-actuated valve-in-line between the aspiration catheter and the vacuum pump;
opening the vacuum-actuated valve in response to the applied vacuum generating a vacuum level at a vacuum-actuated valve that is equal to or exceeds a predetermined vacuum threshold to initiate aspiration of material in the blood vessel via the aspiration catheter; and
during the aspiration, closing the vacuum-actuated valve in response to the vacuum level at the vacuum-actuated valve decreasing below the predetermined vacuum threshold to stop the aspiration of material in the blood vessel via the aspiration catheter.

14. The intravascular treatment aspiration method of claim 13, further comprising:
regulating the aspiration by adjusting a flow of the material using the vacuum-actuated valve-based on the vacuum level at the vacuum-actuated valve.

15. The intravascular treatment aspiration method of claim 13, further comprising:
during the aspiration, collecting the aspirated material in a collection container in-line between the vacuum-actuated valve and the vacuum pump.

16. The intravascular treatment aspiration method of 13, further comprising:
during the aspiration, collecting the aspirated material in a collection container in-line between the aspiration catheter and the vacuum-actuated valve.

17. The intravascular treatment aspiration method of claim 13, wherein vacuum-actuated valve comprises:
a flow inlet operatively connected with the aspiration catheter,
a flow outlet operatively connected with the vacuum pump, and
a vacuum pull actuator operatively connected to the flow outlet whereby vacuum at the flow outlet operates the vacuum pull actuator to open the normally closed flow control valve.

18. The intravascular treatment aspiration method of claim 17, wherein the vacuum pull actuator-includes:
a piston cylinder;
a piston assembly including a piston disposed in the piston cylinder and a piston rod-connected with the piston, the piston rod having a through-hole; and
a spring arranged to bias the piston assembly to a closed position in which the through-hole of the piston rod is misaligned with a fluid flow path from the flow inlet to the flow outlet.

19. The intravascular treatment aspiration method of claim 18, wherein the piston cylinder-is operatively connected to the flow outlet, whereby vacuum at the flow outlet moves the piston assembly to an open position in which the through-hole of the piston rod-is aligned with the fluid flow path.

20. The intravascular treatment aspiration method of claim 18, wherein the through-hole of the piston rod has a teardrop shape and a tip of the teardrop is aligned with the fluid flow path when the piston assembly is in the closed position whereby a nonzero trickle flow path from the flow inlet to the flow outlet is provided in the closed position of the normally closed flow control valve.
